(19) United States
(12) Patent Application Publication
(10) Pub. No.: US 2006/0293556 A1
(43) Pub. Date: Dec. 28, 2006

(54) Endoscope with remote control module or camera

(76) Inventor: David M. Garner, Vancouver (CA)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(21) Appl. No.: 11/130,320

(22) Filed: May 16, 2005

(51) Int. Cl. A61B 1/00

(52) U.S. Cl. 600/101

(57) ABSTRACT

An endoscope with image-capture device remote from the probe is disclosed. The endoscope assembly has a probe, an illumination source such as one or more LEDs, and an image capture device located remotely from the probe such that the user does not have to bear the weight of the image capture device. In one embodiment the image-capture device is located in a control module with an image processor.

### BACKGROUND OF THE INVENTION

This invention relates to the field of endoscopy and more particularly in vivo imaging of cells, tissue, organs or body cavities of humans or other animals to observe, locate, diagnose, and treat disease. Generally, endoscope assemblies have at least one illumination source, the endoscopy device, and an image observation means such as an ocular or an image-capture device such as a camera. In addition, computer processing and data display options are common.

Imaging is the capture of electromagnetic radiation either reflected or emitted from an object of interest such as cells, tissues, organs, or body cavities of humans or other animals, in a manner which preserves or otherwise represents the spatial distribution of said radiation at the object. In the field of medical imaging, and more particularly endoscopy, visible light is utilized to illuminate body tissues and return a diagnostic or otherwise useful image. So, endoscopes enable visual examination of structure inside cavities. The use of endoscopes permits inspection of organs or tissue for the purposes of diagnosis, viewing a surgical site, sampling, or facilitating the safe manipulation of other surgical instruments.

Historically, physicians viewed white light reflectance color images through an ocular attached to the endoscope. More recently, with cost reductions and other computer advances, rather than projecting through an ocular, endoscopy images are captured by a light transducer such as a digital camera and displayed on a monitor. Bronchoscopy serves as an example of a specific endoscopy procedure, in this instance for examining the lungs and respiratory tract. When white light is used for tissue illumination it provides visual indication of the physical structure morphological image of the lungs and bronchial passages. In use, physicians may detect various diseases such as lung cancer by observing features in white light reflectance images such as the color and surface morphology of lung tissue and its various structures.

White light means a broad spectrum or combination of spectra in the visible range. For endoscopy, LEDs, lamps, and lasers, alone or in combination, along with optical elements such as lenses, filters, filter wheels, liquid-crystal filters, and multi-mirror devices, are used to provide the desired white-light illumination. It is considered advantageous for the physician to be presented with a white-light image in real-time at video rate. At the same time as images are displayed, they may be captured and analyzed by computer to extract various features.

Medical research indicates that cancer can be treated more effectively when is detected early when lesions are smaller or when tissue is in a precancerous stage. While changes in the physical appearance morphology and color of tissue using white light are useful, to achieve greater reliability and to detect cancer or other diseases earlier, various endoscopy devices have been developed which have increased sensitivity to the biological composition of tissue. Just as certain morphological changes in tissue may be associated with disease, chemical changes may also be exploited for disease detection, especially for detection of early disease.

One such method of detecting chemical changes in tissue during endoscopic procedure involves utilizing tissue illumination with specific wavelengths or bands of light that interact with certain chemical compounds in tissue, particularly those that are associated with diseases such as cancer. For example, some endoscopy devices utilize light in the UV or UV/blue spectrum to illuminate tissue. These wavelengths of light are selected based on their ability to stimulate certain chemicals in tissue that are associated with disease or disease processes. When illuminated with UV or UV/blue light, tissue may emit light at wavelengths longer than the illumination (also called excitation light) and images or spectra from these fluorescence emissions may be captured for observation and/or analysis. Healthy and diseased tissues fluoresce differently so the spectra of fluorescence emissions can be used as a diagnostic tool.

In addition, to assist in interpreting these fluorescence images, pseudo-colors may be assigned to help visualize the extent and location of diseased tissue. For example, the red color may be assigned to diseased tissue while healthy tissue may be displayed in green. As with any subjective method, standardization and calibration procedures may be used to set color tones or intensities to match image characteristics from instrument to instrument or between devices from different manufacturers.

“Spectroscopy” here refers to analysis of light according to its wavelength or frequency components. The analysis results are usually presented in the form of spectrum or spectra, such as plots of light intensity as a function of wavelength. Reflectance spectroscopy is the analysis of light reflected from the tissue. Biological tissue is a turbid medium, which absorbs and scatters incident light. The majority of the reflected light from tissue has traveled inside the tissue and encountered absorption and scattering events and therefore contains compositional and structural information about the tissue.

Tissue reflectance spectroscopy can be use to derive information about tissue chromophore molecules that absorbs light strongly, e.g. hemoglobin. The ratio of oxy-hemoglobin and deoxy-hemoglobin can be inferred and used to determine tissue oxygenation status which is very useful for cancer detection and prognosis analysis. It can also be used to derive information about scatterers in the tissue such as the size distribution of cell nucleus and average cell density.

Fluorescence spectroscopy is the analysis of fluorescence emission from tissue. Native tissue fluorophore molecules that emit fluorescence when excited by appropriate wavelengths of light include tyrosine, tryptophan, collagen, elastin, flavins, porphyrins, and nicotinamide adenine dinucleotide NAD. Tissue fluorescence is very sensitive to chemical composition and chemical environment changes associated with disease transformation. Fluorescence imaging takes advantage of fluorescence intensity changes in one or more broad wavelength bands thus providing sensitive detection of suspicious tissue areas, while fluorescence spectroscopy especially spectral shape can be used to improve the specificity for detection of early cancer.

Although fluorescence imaging endoscopy provides increased sensitivity to diseases such as cancer, there are also some trade offs. For example, while sensitivity to something abnormal is increased, specificity is reduced, because some non-diseased tissue, e.g. benign tissue, to mimic the chemical signatures of diseased tissue, e.g. cancer, thus making the colored images indistinguishable from true disease. These additional suspect tissue site false positives may require further investigation to confirm disease status; for example, the clinician may need to take a biopsy for examination by a pathologist. Another limitation of fluorescence imaging endoscopy is that, due to the very weak intensity of the emitted light, it does not provide the same image quality for morphological structure and therefore typically requires additional caution and time to guide the endoscope during the procedure.

A typical endoscope has at least one light source to provide interrogating radiation usually white light or blue/ultraviolet light. One typical light source, for example, a xenon lamp, emits white light to produce images of the target object from light reflected or scattered from the target. Another typical light source, for example, a laser operating in a narrow band of the blue or ultraviolet region of the spectrum, emits excitation light to produce images of the target from green-shifted fluorescence from the target. Please note that other light sources are also used as interrogating radiation, including for example, red and near-infrared light sources. Light is generated by the one or more light sources. An interrogating light guide, typically one or more optical fibers, channels the interrogating light to the target object. It interacts with the target object to produce returning radiation in the form of reflectance, absorption, scattering, fluorescence, or Raman spectra. The returning radiation is gathered by various optical lenses and returned the length of the endoscope, typically in a bundle of optical fibers. An interface device such as a switch can be used for selecting the various illumination sources and/or corresponding plurality of returning spectral segments.

In the past, the health-care provider viewed this returning light through an ocular device. The eye has a high brightness and color dynamic range, which has the result that the attainable image quality with respect to the definition and color representation is almost entirely dependent on the endoscope employed. Advances in electronic technology, principally charge-coupled devices, allows coupling of the returning light fiber optic bundle to a detector such as a camera, whereupon the images can be viewed on a monitor. And, since CCD cameras produced digital images, those images can be processed in various ways before display on a monitor.

Other advances in electronics have allowed the placement of light sources near the distal end of the endoscope. For example, by placing LEDs at the end of the endoscope, the fiber optics carrying the illumination or excitation light can be eliminated. LEDs are lower cost, more reliable, longer lasting, lighter in weight, more compact and more efficient than lasers and lamp sources, allowing for better control of imaging and illumination. Moreover, LEDs can be switched quickly, allowing for time-gated studies, improved disease detection sensitivity, and increased disease detection specificity. Additionally, eliminating the interrogating radiation fiber optics confers certain benefits including lower cost, simplified assembly, increased reliability, improved flexibility and decreased size.

An endoscope 112 as is known in the prior art, as shown in FIG. 1, has a probe 114 which is an elongated tube tapering to a channel 120 at its distal end. Channel 120 is adapted for insertion into the patient for whom an examination for disease or diagnosis of disease is desired. Probe 114 usually contains a switching mechanism for the user to toggle between various illuminating and/or display sources, and a steering mechanism that rotates the distal tip of channel 120 as the user navigates the distal tip through a patient's body cavity or surgical incision.

Channel 120 contains at least one light guide 124, which is typically an optical fiber, to bring interrogating radiation to the target, and an imaging bundle, which is typically at least one optical fiber and usually is many optical fibers, to bring returning radiation away from the target. A light source 116 generates interrogating radiation, which can be some combination of white light for color images, narrow-band excitation light for fluorescence, other narrow-band light for normalization, or other types of light. The interrogating radiation travels through light guide 124, through probe 114 and channel 120, and is incident on the target, typically tissue within a body cavity or surgical incision of the patient. Returning radiation, which can be reflected white light, reflected excitation light, scattered white or excitation light, or fluorescence light, is collected by various lenses into the imaging bundle 126 and conducted through channel 120 and probe 114 to image-capture device 136, typically a camera.

Image-capture device 136 is usually located integral to or immediately proximal to probe 114. Accordingly, the user has to carry the weight of the camera 136 in addition to the weight of probe 114 while manipulating the probe 114 during an endoscopy procedure. In some instances, there may be a tether supporting the weight of the probe and camera, but the user still has to contend with the mass of the combined devices.

The distal end of channel 120 of FIG. 1 is shown in more detail in FIG. 2. Channel 220, as shown in cross-section in FIG. 2, typically contains one or more fiber optic light guides 224 to carry the interrogating radiation to the target object (such as tissue) and an imaging bundle 226 to carry imaging radiation from the tissue. Channel 220 also contains an instrument channel 228 for biopsy or other surgical procedures, a water tube 230 for lavage of the target, and an air tube 232 for suction. In addition the instrument channel of an endoscope may provide access for micro-surgery devices, releasing nano-devices, optical computed tomography, confocal microscopy, laser or drug treatments, gene-therapy, injections, marking, implanting, or other medical procedures. Channel 220 has an outer layer 234, usually made of plastic that can be sterilized, to hold the various components together.

Other recent innovations include placing miniature image-capture devices at the distal end of the endoscope. This configuration eliminates the need for fiber optic bundle to channel the returning radiation to a camera. Instead, the miniature image-capture device sends signals to a processor such a computer. This configuration provides an opportunity for increased resolution and improved imaging. As stated above, eliminating the fiber optic bundle for the returning radiation allows for lower cost, simplified assembly, increased reliability, improved flexibility and decreased size. However, these advantages must be weighed against the disadvantages of increased cost for miniature image-capture device, which tends to cost more than a conventionally-sized image-capture device.

Additionally, whether the image-capture device is placed at the distal end of the probe or at the proximal end of the probe as is done in the prior art, the image-capture device tends to get bumped about during use. Regardless of the type or size of image-capture device used, such as a conventional camera, a digital camera, or a spectrometer, rough handling is usually detrimental to the device.

Using current technology, the video tower takes up significant space near the patient and the operating room staff. The mass of the equipment that is necessary to create and display the images and their proximity to the operative site and the location and number of interconnecting elements make the endoscope hard to manage.

Because of these drawbacks in the traditional video endoscopy systems, a need exists for an endoscope that would take equipment out of the footprint of the operative area and that a physician can operate with increased freedom of movement and less fatigue. The present invention meets this need.

### BRIEF SUMMARY OF THE INVENTION

It is an object of the present invention to provide an endoscope for imaging interior body parts of patients, with augmented image fiber bundle that does not terminate at the ocular. In another embodiment, an imaging device such as a camera is relocated into the control module. The control module is connected with the endoscope probe through the channel, which contains a light guide that carries the interrogating radiation to the target, and an image bundle that carries imaging radiation from the target. In one embodiment the endoscope contains illumination components mounted at the distal end of the endoscope. In the other embodiment the endoscope contains an image-capture device disposed at the distal end of the endoscope capable of operating in one mode, and another image-capture device attached with augmented image fiber optic to an external portion outside the endoscope's body capable of operating in another mode.

The endoscope of the present invention is easier to operate. Since the camera is not mounted or attached to the endoscope, the physician can operate the device with increased freedom of movement and less fatigue.

### BRIEF DESCRIPTION OF THE SEVERAL VIEWS OF THE DRAWINGS

The organization and manner of the structure and operation of the invention, together with further objects and advantages thereof, may best be understood by reference to the following description, taken in connection with the accompanying drawings, in which:

FIG. 1 is an illustration of an endoscope as is known in the prior art.

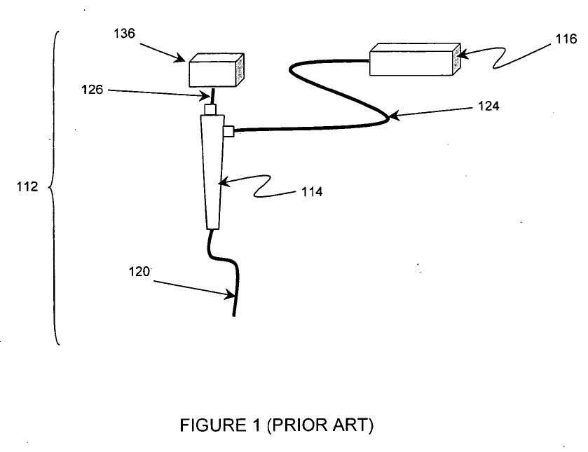

FIG. 2 is a cross-sectional view of a typical channel of an endoscope as is known in the prior art.

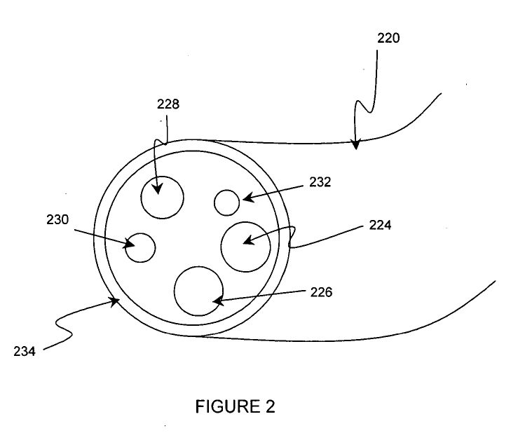

FIG. 3 is a cross-sectional view of an embodiment of an endoscope apparatus of the present invention.

FIG. 4 is an illustration of another embodiment of an endoscope of the present invention.

FIG. 5 is an illustration of yet another embodiment of an endoscope of the present invention.

### DETAILED DESCRIPTION OF THE PREFERRED EMBODIMENTS OF THE INVENTION

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that embodiment as illustrated and described herein.

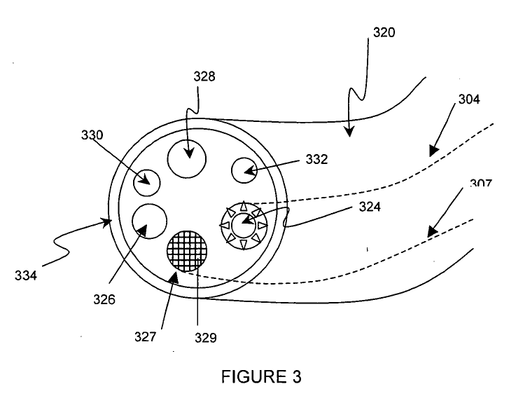

One embodiment of the present invention is shown in FIG. 3. In this embodiment, illumination components are placed at the distal end of the channel 320, as shown in cross-section in FIG. 3. Illumination source 324, located at the distal end of channel 320, generates desired interrogating radiation onto the target tissue. Illumination source 324 is preferably at least one LED, more preferably at least two LEDs. Illumination source 324 could also be a laser, a xenon lamp, a mercury lamp, tungsten halogen lamp, metal halide lamp or other light source. Power and control of illumination source 324 are provided through wire 304, which couples of illumination source 324 to a standard power source and its control electronics, not shown.

Interrogating light generated by illumination source 324 is incident on the target, which produces reflected light, scattered light, and fluorescence light, as discussed above. This returning light is gathered by objective lens 329, which preferably has shielding filters, and is captured by image-capture device 327, typically a camera, such as a CCD array. Power and control of image-capture device 327 are provided through wire 307, which couples image-capture device 327 to a standard power source, not shown, and to an image display or an image processor, or both. For example, wire 307 carries signals corresponding to the returning light back to a monitor for display and to a computer for image processing, or to both. Both the image display and the image processor are located remote from the probe, meaning neither the user nor a tether carries the weight of the image display or image processor.

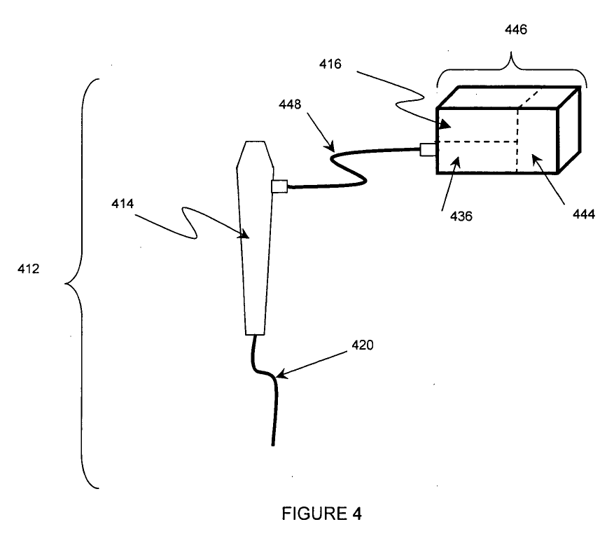

This embodiment can also have a portion of the returning radiation gathered into optical fiber 326 through a filter or an orifice in the fiber mirror. This part of the returning radiation through optical fiber 326 is carried through channel 320 and directed to a detector, such as image-capture device 436 as will be described in connection with FIG. 4. The detector can be a camera, a spectrometer, or another image-capture or image-processing device, and is also located remote from the probe. Processing of returning radiation through two modalities, such as white-light imaging and fluorescence imaging, or imaging and spectrometry, is described in, for example, U.S. patent application Ser. No. 10/431,939, Real-Time Contemporaneous Multimodal Imaging and Spectroscopy Uses Thereof”, Published Patent Application No. 2004/0225222 A1, the disclosure of which is incorporated herein by reference, which discusses various devices and configurations for simultaneous white-light and fluorescence imaging.

Signals from both detectors 327, 436 are delivered to the processing unit, as will be described in connection with FIG. 4, which can be a microprocessor, a computer, a collection of integrated circuits, a collection of analog circuits, and/or a spectrometer. The processing unit processes the signals and creates color images, false color images, pseudo-images, normalized images and/or spectrograms of the imaging radiation. Representative processing techniques are described in, for example, U.S. patent application Ser. No. 10/431,939, described above.

The channel 320 may also contains an instrument channel 328 for biopsy or other medical procedures, such as optical computed tomography, confocal microscopy, laser or drug treatments, gene-therapy, injections, marking, implanting or other medical techniques. In addition the channel 320 contains a water tube 330 for lavage of the target and an air tube 332 for suction. An outer layer 334, usually plastic holds the various components together.

An endoscope assembly 412 of a preferred embodiment of the present invention is shown in FIG. 4, in which the illumination source, image-capture device, and processor are contained in an integral unit. Endoscope 412 has a probe 414 which is an elongated tube having a channel 420 at the distal end. Channel 420 is adapted for insertion into the patient for whom an examination for disease, screen for disease, diagnosis of disease, or surgical procedure is desired. Channel 420 contains at least one light guide, preferably an optical fiber, to bring interrogating radiation to the target, and an imaging bundle, preferably at least one optical fiber and more preferably many optical fibers, to bring returning radiation away from the target.

A second channel 448 extends from near the proximal end of probe 414 to control module 446 and contains the light guide and an imaging bundle. The control module 446 includes an illumination source 416, an image-capture device 436, and a processing unit 444.

Illumination source 416 is an LED, several LEDs, a laser, a xenon lamp, a mercury lamp, a tungsten halogen lamp, another metal halide lamp; or other light source. Illumination source 416 generates interrogating radiation, which can be some combination of white light for color images, narrow-band excitation light for fluorescence, other narrow bands for normalization, or other types of light. The interrogating radiation is carried by the one or more light guides through second channel 448, through probe 414, and through channel 420 to a target, which will reflect, scatter, or fluoresce the interrogating radiation, depending on what type of light was used to illuminate the target, to produce returning radiation.

Returning radiation, which can be reflected white light, reflected excitation light, scattered white or excitation light, or fluorescence light, is gathered by various lenses into the imaging bundle and is channeled through channel 420, probe 414, and second channel 448 to the image-capture device 436. An interface device such as a switch mounted on probe 414 is preferably used for switching to the various illuminating sources and/or between returning spectral segments of different modalities.

Returning radiation that is captured in the image-capture device 436 is processed in processing unit 444, which can be a microprocessor, a computer, a collection of integrated circuits, a collection of analog circuits, and/or a spectrometer. Processing unit 444 can be used to create color images, false color images, pseudo-images, normalized images, and spectrograms of the imaging radiation. Various filters can intercept the imaging radiation en route to the image-capture device 436 to enhance the ability to create these images or spectrograms. Processing unit 444 can send its output to one or more monitors for visualization by humans, to printers for permanent records, or to a database software program for statistical analysis. U.S. patent application Ser. No. 10/431,939, Real-Time Contemporaneous Multimodal Imaging and Spectroscopy Uses Thereof”, Published Patent Application No. 2004/0225222 A1, the disclosure of which is incorporated herein by reference, describes techniques for such processing, and is incorporated herein by reference.

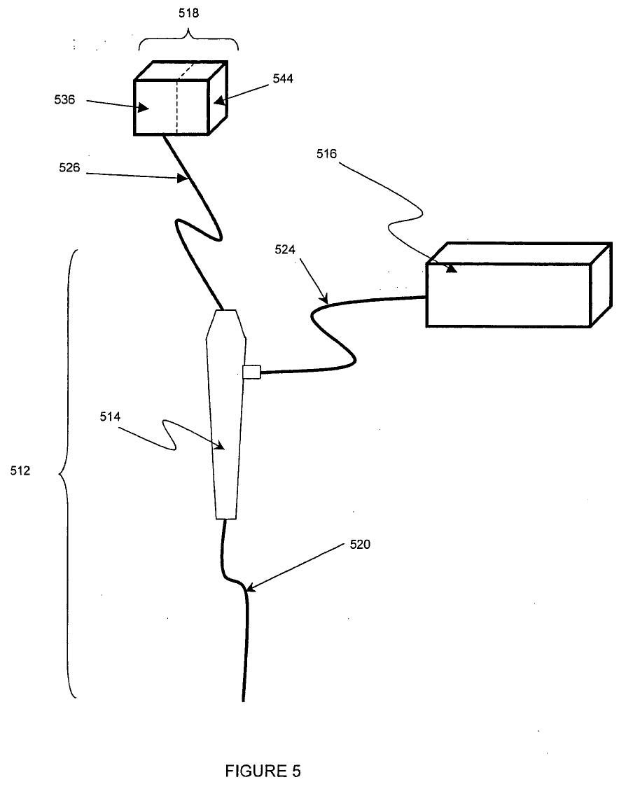

In yet another embodiment of the present invention, the illumination source is positioned separately from the image-capture device and processing unit. As shown in FIG. 5, endoscope assembly 512 has a probe 514, a light source 516, and a control unit 518. Control unit 518 contains image-capture device 536, preferably a camera such as a CCD or similar device, and processing unit 544. Control unit 518 is remote from probe 514, so that neither a user of the endoscope assembly 512 nor a tether holding up probe 514 bears the weight of control unit 518.

As with other endoscopes, probe 514 is an elongated tube tapering to a channel 520 and is adapted for insertion into the patient for whom an examination for disease or diagnosis of disease is desired. Light source 516 is an LED, two or more LEDs, a laser, a xenon lamp, a mercury lamp, a tungsten halogen lamp, another metal halide lamp, or other light source. Light source 516 generates interrogating radiation, which can be some combination of white light for color images, narrow-band excitation light for fluorescence, other narrow bands for normalization, or other types of light. The interrogating radiation travels through light guide 524 and channel 520 and it is incident on the target. Returning radiation, which can be reflected white light, reflected excitation light, scattered light, or fluorescence light, is collected by various lenses into imaging bundle 526 and conducted through channel 520 and probe 514 to image-capture device 536. An interface device such as a switch mounted on probe 514 is preferably used for switching to the various illuminating sources and/or between returning spectral segments of different modalities.

Returning radiation that is captured in the image-capture device 536 is processed in processing unit 544, which can be a microprocessor, a computer, a collection of integrated circuits, a collection of analog circuits, and/or a spectrometer. Processing unit 544 can be used to create color images, false color images, pseudo-images, normalized images and spectrograms of the imaging radiation. Processing unit 544 sends its output to one or more monitors for visualization by humans, to printers for permanent records or to a database software program for statistical analysis.

While preferred embodiments of present invention are shown and described, it is envisioned that those skilled in the art may devise various modifications of the present invention without departing from the spirit and scope of the claims.

I claim:

1. An endoscope assembly comprising:
a probe;
an illumination source to generate interrogating radiation to illuminate a target object and produce returning radiation;
an imaging bundle to channel said returning radiation through said probe to an image capture device located remote from said probe.

2. The endoscope assembly of claim 1, wherein said interrogating radiation comprises at least one of broadband light and narrow-band light.

3. The endoscope assembly of claim 1, wherein said interrogating radiation comprises light in a first spectrum comprising narrow-band light to produce fluorescence and light in a second spectrum comprising at least one of a broadband spectrum for normalization and a narrow-band spectrum for normalization.

4. The endoscope assembly of claim 1, wherein said illumination source is located in the distal end of said probe.

5. The endoscope assembly of claim 1, wherein said illumination source is located remote from said probe and further comprising a light guide to channel said interrogating radiation through said probe to said target object.

6. The endoscope assembly of claim 1, further comprising an image display coupled to said image-capture device.

7. The endoscope assembly of claim 6, wherein said image display produces at least one of a color image, a false color image, a pseudo-image, a normalized image, and a spectrogram.

8. The endoscope assembly of claim 1, further comprising an image processor coupled to said image capture device.

9. The endoscope assembly of claim 1, wherein said image processor comprises at least one of a microprocessor, a computer, an integrated circuit, a plurality of integrated circuits, a plurality of analog circuits, and a spectrometer.

10. The endoscope assembly of claim 1, further comprising image-processing means.

11. The endoscope assembly of claim 1, further comprising an image display and an image processor coupled to said image capture device.

12. The endoscope assembly of claim 1, wherein said probe further comprises at least one of an instrument channel, a water tube, and an air tube.

13. The endoscope assembly of claim 1, wherein said light-source means comprises at least one of an LED, a plurality of LEDs, a laser, a xenon lamp, a mercury lamp, a tungsten halogen lamp, and a metal halide lamp.

14. The endoscope assembly of claim 1, further comprising a detector located remote from said probe and said imaging bundle further channels at least a portion of said returning radiation to said detector.

15. The endoscope assembly of claim 14, wherein said detector comprises at least one of a camera and a spectrometer.

16. An endoscope assembly comprising:
a probe;
an illumination source to generate interrogating radiation to illuminate a target object and produce returning radiation;
an image-capture device to capture said returning radiation;
imaging means remote from said probe and coupled to said image-capture device to display images of said returning radiation.

17. The endoscope assembly of claim 16, wherein said interrogating radiation comprises at least one of broadband light and narrow-band light.

18. The endoscope assembly of claim 16, wherein said interrogating radiation comprises light in a first spectrum comprising narrow-band light to produce fluorescence and light in a second spectrum comprising at least one of a broadband spectrum for normalization and a narrow-band spectrum for normalization.

19. The endoscope assembly of claim 16, wherein said illumination source is located in the distal end of said probe.

20. The endoscope assembly of claim 16, wherein said illumination source is located remote from said probe and further comprising a light guide to channel said interrogating radiation through said probe to said target object.

21. The endoscope assembly of claim 16, further comprising an image processor coupled to said image capture device.

22. The endoscope assembly of claim 21, wherein said image processor comprises at least one of a microprocessor, a computer, an integrated circuit, a plurality of integrated circuits, a plurality of analog circuits, and a spectrometer.

23. The endoscope assembly of claim 16, further comprising image-processing means.

24. The endoscope assembly of claim 16, further comprising and an image processor coupled to said image capture device.

25. The endoscope assembly of claim 16, wherein said probe further comprises at least one of an instrument channel, a water tube, and an air tube.

26. The endoscope assembly of claim 16, wherein said light-source means comprises at least one of an LED, a plurality of LEDs, a laser, a xenon lamp, a mercury lamp, a tungsten halogen lamp, and a metal halide lamp.

27. The endoscope assembly of claim 16, further comprising a detector located remote from said probe and said imaging bundle further channels at least a portion of said returning radiation to said detector.

28. The endoscope assembly of claim 27, wherein said detector comprises at least one of a camera and a spectrometer.

29. The endoscope assembly of claim 16, wherein said images comprise at least one of a color image, a false color image, a pseudo-image, a normalized image, and a spectrogram.

30. An endoscope assembly comprising:
a probe;
a control module located remote from said probe;
an illumination source to generate interrogating radiation to illuminate a target object and produce returning radiation;
an imaging bundle to channel said returning radiation through said probe to said control module.

31. The endoscope assembly of claim 30, wherein said interrogating radiation comprises at least one of broadband light and narrow-band light.

32. The endoscope assembly of claim 30, wherein said interrogating radiation comprises light in a first spectrum comprising narrow-band light to produce fluorescence and light in a second spectrum comprising at least one of a broadband spectrum for normalization and a narrow-band spectrum for normalization.

33. The endoscope assembly of claim 30, wherein said illumination source is located in the distal end of said probe.

34. The endoscope assembly of claim 30, wherein said illumination source is remote from said probe and said endoscope assembly further comprises a light guide to channel said interrogating radiation through said probe to said target object.

35. The endoscope assembly of claim 30, wherein said control module comprises an image-capture device, an image processor, and said illumination source.

36. The endoscope assembly of claim 35, wherein said image processor comprises at least one of a microprocessor, a computer, an integrated circuit, a plurality of integrated circuits, a plurality of analog circuits, and a spectrometer.

37. The endoscope assembly of claim 30, wherein said control module comprises an image-capture device and an image processor.

38. The endoscope assembly of claim 37, wherein said image processor comprises at least one of a microprocessor, a computer, an integrated circuit, a plurality of integrated circuits, a plurality of analog circuits, and a spectrometer.

39. The endoscope assembly of claim 30, wherein said control module comprises an image-capture device and image-processing means.

40. The endoscope assembly of claim 30, wherein said probe further comprises at least one of an instrument channel, a water tube, and an air tube.

41. The endoscope assembly of claim 30, wherein said light-source means comprises at least one of an LED, a plurality of LEDs, a laser, a xenon lamp, a mercury lamp, a tungsten halogen lamp, and a metal halide lamp.

42. The endoscope assembly of claim 30, wherein said control module further comprises a detector and said imaging bundle further channels at least a portion of said returning radiation to said detector.

43. The endoscope assembly of claim 42, wherein said detector comprises at least one of a camera and a spectrometer.

44. The endoscope assembly of claim 30, further comprising imaging means to display images of said returning radiation.

45. The endoscope assembly of claim 44, wherein said images comprise at least one of a color image, a false color image, a pseudo-image, a normalized image, and a spectrogram.
